(19) United States
(12) Patent Application Publication
(10) Pub. No.: US 2021/0262903 A1
(43) Pub. Date: Aug. 26, 2021

(54) NON-DESTRUCTIVE SAMPLING SYSTEM AND METHOD FOR QUALITY ASSESSMENT OF BLOOD PRODUCTS, AND SAMPLING SYSTEMS THEREFOR

(71) Applicant: CANADIAN BLOOD SERVICES, Ottawa (CA)

(72) Inventors: Peter SCHUBERT, Richmond (CA); Ken MCTAGGART, Ottawa (CA)

(21) Appl. No.: 17/059,006

(22) PCT Filed: May 31, 2019
(86) PCT No.: PCT/CA2019/050758

§ 371 Date: Nov. 25, 2020

Related U.S. Application Data
(60) Provisional application No. 62/679,264, filed on Jun. 1, 2018.

(51) Int. Cl. G01N 1/10; G01N 1/38

(52) U.S. Cl. CPC G01N 1/10; G01N 2001/387; G01N 1/38

(57) ABSTRACT

There is provided a method of sampling an aliquot of blood products from a main container containing the blood products to be sampled. The method includes providing a sample container that is rectangular-shaped and has a length-over−width (L/W) ratio of at least that of the main container, and fluidly connecting the a sample container to the main container, transferring the aliquot of the blood products from the main container to the sample container, and forming an air bubble in the sample container by introducing a volume of air into the sample container with the aliquot of the blood products. The volume of air forming the air bubble corresponds to at least about 5% of a volume of the aliquot of the blood products. A sampling bag and a sampling system are disclosed.

### TECHNICAL FIELD

The present application relates generally to systems and methods for storing blood products for later time point analysis and, more particularly, to systems and methods for sampling blood products for analysis purposes.

### BACKGROUND

Blood products are living biological substances donated by donors for later use in the medical treatment of patients, for example those who are in need of a transfusion. The whole blood is removed from their natural environment in the body, processed to remove or separate the whole blood into specific blood components, then stored in dedicated containers, typically plastic storage bags, and in controlled environments to minimize deleterious damage over time. The blood products may be transfused into a patient where they must effectively resume their biological function. As is well known in the field, the collection, manufacturing, and storage processes used to produce blood products for transfusion purposes all contribute to the type and rate of deterioration during the ‘storage lesion’ that spans from the time of collection to the time of transfusion. The storage lesion may be affected by individual donor characteristics, also referred to as ‘donor factors’.

It is a challenge for blood bankers to ensure safety and effectiveness of products throughout their shelf life. At present, the primary method of process monitoring and control used by blood banks consists of quality attribute process control testing on a number of units selected and held to expiry. Such a process is ‘destructive’ in nature since the units must be held to expiry before they can be sampled for quality attribute testing, and as such cannot be subsequently used for transfusion to patients in need.

In many jurisdictions around the world, sampling of units at expiry for process control is a requirement imposed on blood bankers by various local, national, and international regulations and standards. The number of units selected for testing/sampling, often referred to as the process control sample size, defined by such regulations and standards is however not harmonized between jurisdictions. In general, typical process control sample sizes correspond to around 1% of each of the blood components (e.g., red blood cells, platelets, plasma) of the total product collected. However, such a sample size was historically chosen for pragmatic reasons, to minimize the loss of product, and was not selected on a statistical basis where the sample size would be chosen to provide a high level of confidence that the quality attributes derived from the sampled units are representative of the entire population of units from which the sample was taken.

### SUMMARY OF THE INVENTION

There is generally provided a non-destructive sampling system and method for quality assessment of blood products.

In one aspect, there is provided a method of sampling an aliquot of blood products from a main container containing the blood products to be sampled, the method comprising: providing a sample container that is rectangular-shaped and has a length-over-width (L/W) ratio of at least that of the main container, and fluidly connecting the sample container to the main container; transferring the aliquot of the blood products from the main container to the sample container; and forming an air bubble in the sample container by introducing a volume of air into the sample container with the aliquot of the blood products, the volume of air forming the air bubble corresponding to at least about 5% of a volume of the aliquot of the blood products.

In a particular embodiment, the method further includes using a tube to fluidly connect the sample container to the main container, wherein introducing the volume of air includes injecting air contained within the tube into the sample container.

In a particular embodiment, injecting the air includes transferring the air with the blood products from the main container to the sample container via the tube.

In a particular embodiment, introducing the volume of air includes anchoring the sample container at a given distance from a docking location at which the sample container is fluidly connected to the main container via the tube.

In a particular embodiment, the method further includes fluidly connecting the tube to the sample container.

In a particular embodiment, anchoring the sample container at the given distance includes inserting at least one locking pin secured to a bench supporting the sample container inside at least one aperture defined through a flange of the sample container.

In a particular embodiment, inserting the at least one locking pin through the at least one aperture includes inserting two locking pins through two apertures defined through the flange.

In a particular embodiment, transferring the aliquot of the blood products includes filling the sample container until the sample container becomes compressed between two spaced apart plates.

In a particular embodiment, the method further includes disposing the sample container between the two spaced apart plates.

In a particular embodiment, the method further includes determining a correlation between a distance between the two spaced apart plates and a maximum volume of fluid containable within the sample container located between the two spaced apart plates.

In a particular embodiment, transferring the aliquot of the blood products includes drawing the blood products out of the main container by gravity.

In a particular embodiment, the method further includes hanging the main container above the sample container.

In a particular embodiment, the method further includes mixing the extracted portion of the blood products in the sample container using the air bubble.

In a particular embodiment, the blood products are blood platelets, transferring the extracted portion includes transferring the extracted aliquot in the sample container having the length-over-width (L/W) ratio of about 3.

In a particular embodiment, introducing the volume of air in the sample container includes providing the sample container with the volume of air therein before transferring the extracted aliquot in the sample container.

In a particular embodiment, the method further includes moving the at least one air bubble within the sample container by agitating the sample container.

In a particular embodiment, transferring the aliquot of the blood products includes extracting at most 20% of a volume of the blood products contained within the main container.

In a particular embodiment, the blood products are blood platelets, providing the volume of air includes providing the volume of air corresponding to at least 15% of the volume of the extracted aliquot of the blood products.

In another aspect, there is provided a sampling bag for holding blood products comprising: a sample container defining therein an internal volume adapted to contain the blood products, the sample container made of a flexible plastic film and having a rectangular shape defined by short sides and long sides, the short sides and the long sides delimiting the internal volume of the container, the long sides having a length (L) and the short sides having a width (W), wherein a ratio (L/W) of the length over the width is at least 1.5; and at least one port secured to one of the long sides or to one of the short sides, the at least one port fluidly connectable to a source of the blood products for inserting the blood products into the internal volume of the container.

In a particular embodiment, the ratio (L/W) is at most 10.

In a particular embodiment, the blood products are platelets, and the ratio (L/W) is about 3.

In a particular embodiment, the plastic film is made of plasticized polyvinyl chloride.

In a particular embodiment, the blood products are platelets, and a plasticizer of the plastic film being a citrate.

In a particular embodiment, the citrate is n-butyryl-tri-n-hexyl citrate.

In a particular embodiment, the sampling bag includes a fixing mechanism for securing the sampling bag to an agitating device.

In a particular embodiment, the internal volume ranges from 5 ml to 25 ml.

In a particular embodiment, the at least one port includes a tube of medical fluid flexible tubing, the tube having a length extending from a first extremity to a second extremity, the first extremity being secured to the container and fluidly connected thereto, the second extremity being configured for being fluidly connected to a main container of the blood products.

In yet another aspect, there is provided a sampling system for sampling blood products, comprising: a main container configured for containing a first volume of the blood products; a sampling bag having a sample container made of a plastic film and configured for containing a second volume of the blood products, the sample container having a rectangular shape and having a length-over-width (L/W) ratio of at least that of the main container, the second volume being less than the first volume; and a fluid connection between the main container and the sample container.

In a particular embodiment, the sampling system further includes a filling device for measuring the second volume, the filling device including two places being spaced apart from one another by a gap, the sample container within the gap, a maximum volume of fluid contained in the sample container being defined by a height of the gap.

In a particular embodiment, the filling device includes a support arm extending transversally relative to the plates, the support arm having a holder above the spaced apart plates for holding the main container above the sample container.

In a particular embodiment, the sampling system further includes an apparatus for measuring a volume of air to be inserted in the sample container, the apparatus having: a bench supporting the sample container; a sterile docking device for fluidly connecting the sample container to the main container via a tube; and at least one pin protruding from the bench, the at least one pin received within at least one aperture defined through a flange of the sample container, wherein a distance along a length of the tube from the at least one locking pin to a docking location of the sterile docking device is selected such that a volume of air contained within the tube along the distance corresponds to the volume of air to be inserted in the sample container.

In a particular embodiment, the blood products are blood platelets, the ratio of the length over the width being about 3.

In a particular embodiment, the plastic film is made of plasticized polyvinyl chloride.

In a particular embodiment, a ratio of the second volume over the first volume is at most 0.2.

More particularly, there is provided a sampling bag for holding blood products, the sampling bag comprising a sealable, closed, and sterile container defining therein an internal volume adapted to contain the blood products, the container made of a flexible plastic film and having a rectangular shape defined by short sides and long sides, the short sides and the long sides delimiting the internal volume of the container, the long sides having a length (L) and the short sides having a width (W), wherein a ratio (L/W) of the length over the width is greater than 1.5, and at least one port secured to one of the long sides or to one of the short sides, the at least one port operable to introduce the blood products into the internal volume of the container and for subsequent removal for analysis at a later time.

There is also provided a method of sampling an aliquot of blood products from a main container containing the blood products to be sampled, comprising: fluidly connecting a sample container to the main container, the sample container having a rectangular shape with a length (L) and a width (W), wherein a ratio of the length over the width is greater than 1.5; extracting the aliquot of the blood products from the main container and transferring the extracted aliquot into the sample container; providing a volume of air in the sample container with the extracted portion of the blood products to form an air bubble in the sample container, the air bubble having a predetermined volume; and using the air bubble for mixing, in the sample container, the aliquot of the blood products.

There is further provided a sampling system for sampling blood products, comprising: a main container configured for containing a first volume of the blood products; a sampling bag having a sample container made of a plastic film and configured for containing a second volume of the blood products, the sample container having a rectangular shape and having a length (L) and a width (W), a ratio of the length over the width being greater 1.5, the second volume being less than the first volume; and a fluid connection between the main container and the sample container.

### BRIEF DESCRIPTION OF THE DRAWINGS

Reference is now made to the accompanying figures in which:

FIG. 1 is a schematic top view of a sampling system in accordance with one embodiment;

FIG. 2 is a schematic tridimensional view of a sampling bag of the system of FIG. 1 secured to an agitating device;

FIG. 3 is a schematic tridimensional view of a measuring device in accordance with one embodiment;

FIG. 4 is a schematic tridimensional view of a portion of the measuring device of FIG. 3;

FIG. 5 is a schematic top view of a measuring jig in accordance with one embodiment;

FIG. 6a is a schematic tridimensional view of an integrated sterile docking device with an apparatus to measure a volume of air in accordance with one embodiment;

FIG. 6b is a schematic tridimensional view of the integrated sterile docking device with an apparatus to measure a volume of air in accordance with another embodiment; and

FIG. 7 is a schematic tridimensional view of the sampling system of FIG. 1 with a blocking device in accordance with one embodiment.

### DETAILED DESCRIPTION

The term “blood products” as used herein is understood to refer to any type of blood and/or partial blood product or component, including for example whole blood, leukoreduced whole blood, red cell concentrates, platelet concentrates, pooled platelet concentrates, platelets, red cells, plasma and/or any combination thereof. In some cases, these products are derived from a whole blood donation collected into a special collection set and subsequently manufactured into specific products or components. In other cases, these products are collected directly from a donor using a special apheresis collection machine designed to collect one or more products from the donor's blood and to return the unneeded products to the donor.

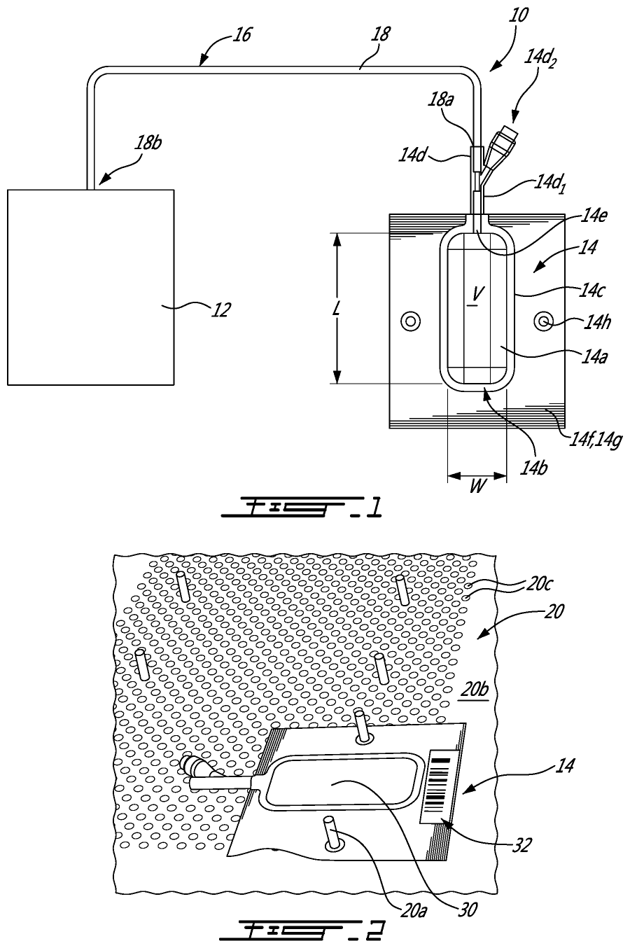

Referring now to FIG. 1, a sampling system for sampling blood products is generally shown at 10. The sampling system 10 includes a main container 12 made of a plastic film and configured for containing a first volume of the blood products and a sampling bag 14 having a sample container 14a being sterile and made of a plastic film and configured for containing a second volume of the blood products; the second volume being less than the first volume. The sampling bag 14 is used to contain a small portion, or aliquot, of the blood products that is extracted from the main container 12 for analysis purposes. In the embodiment shown, and when the blood product is platelets, a ratio of the second volume of the sample container 14a over the first volume of the main container 12 ranges from 0.0025 to 0.05, and more particularly may range from 0.0025 to 0.015. In one exemplary embodiment, the internal volume of the main container 12 is about 1800 mL.

Table 1 below lists, for a plurality of blood product types, and for a particular embodiment, the volumetric capacity of the main and sample containers, the typical volume of the blood product type that is contained within the main and sample containers, and the ratio of the volume of the product type over the volumetric capacity of the main and sample containers.

TABLE 1

| | | | | | | Ratio of |
|---|---|---|---|---|---|---|
| | | Typical | | Typical | | Product |
| | | Nominal | | Product | | Fill to |
| Product | | Container | | Fill | | Container |
| Type | Container Type | Capacity | | Volume | | Capacity |
| Generic | Main Container | 600-1800 | mL | 150-500 | mL | 0.08-0.2 |
| Blood | Sample | 5-25 | mL | 5-25 | mL | 0.2-1.0 |
| Product | Container | | | | | |
| | Ratio Sample | 0.002-0.04 | | 0.01-0.2 | | |
| | Container to | | | | | |
| | Main Container | | | | | |
| Platelet | Main Container | 1800 | mL | 200-350 | mL | 0.1-0.2 |
| Concentrates | Sample | 17.5 | mL | 12 | mL | 0.7 |
| | Container | | | | | |
| | Ratio Sample | 0.01 | | 0.03-0.06 | | |
| | Container to | | | | | |
| | Main Container | | | | | |
| Red Cell | Main Container | 600 | mL | 300 | mL | 0.5 |
| Concentrates | Sample | 17.5 | mL | 9 | mL | 0.5 |
| | Container | | | | | |

| | | |
|---|---|---|
| Ratio Sample | 0.03 | 0.01 |
| Container to | | |
| Main Container | | |

The sampling bag 14 is configured to be fluidly connected in a sterile manner to the main container 12 of blood products via a fluid connection 16 therebetween. The volume of blood products contained within the main container 12 is greater than that of the sampling bag 14.

As shown, the container 14a has a rectangular shape and has short sides 14b and long sides 14c. The short and long sides 14b, 14c delimit an internal volume V of the container 14b for receiving the blood products. Each of the short sides 14b extends from one of the long sides 14c to the other; each of the long sides 14c extends from one of the short sides 14b to the other. The long sides 14c each have a length L and the short sides 14b each have a width W. The ratio of the length L over the width W is greater than 1.5. The ratio of the length L over the width W is selected depending on which of the blood products is to be contained within the sample container 14b. In a particular embodiment, the blood products are blood platelets; the ratio of the length L over the width W being about 3. In a particular embodiment, the ratio of the length L over the width W ranges from 1.5 to 10. The internal volume V of the container 14b ranges from 5 to 25 ml depending on which blood products is contained therein. In a particular embodiment, the blood products are red blood cells; the ratio of the length L over the width W being about 3.

One would have expected that the optimum length-over-width ratio of the sample container should be the same as that of the main container. That is, that the optimum shape of the sample container would be simply the same shape as the main container, albeit scaled down.

The length-over-width ratio of about 3 for the sample container containing platelets was however unexpectedly found to be optimal by the present inventors. In fact, attempting to replicate the fluid flow in the sample container containing platelets with ratios smaller than 3 was tried and found to be insufficient. In the absence of effective mixing, platelets might not remain suspended in solution and might begin to aggregate and clump.

Having a length-over-width ratio of at least 3 is advantageous since it was unexpectedly found that this was the minimum ratio needed to enable platelet concentrate fluid mixing in the sample containers when placed on the same agitators as the main container. It was found that this ratio enables movement of the air bubble in the sample container which helps fluid flow and effective mixing, thereby minimizing the delta in platelet storage lesion as measured in the sample container as compared to that measured in the main container.

In addition, an air bubble causes foaming and is deleterious to the platelets if contained within the main container. In fact, the main containers containing platelets are “burped” in production to remove any residual air bubbles before being place onto the agitators. Consequently, the utilization of the air bubble in the sample container containing platelets is counter-intuitive.

Red cells are less sensitive to sample container geometry since they do not require agitation during storage, as is the case for platelets. The length-over-width ratio of about 1.5 reflects ‘scaling’ of the main container geometry down to sample container size. For red blood cells, the same length-over-width ratio for both of the main container and the sample container works. However, it was found that a higher length-over-width ratio, about 3, also works. To simplify manufacturing of the containers, the length-over-width ratio of 3 may be used for both red cells and platelets sample containers, since that ratio is required for platelets to get optimal performance but less critical for red cells.

The sampling bag 14 includes at least one port 14d operable to fill and to empty the container 14b of the blood products. In the embodiment shown, the at least one port 14d is located at one of the short sides 14b. As illustrated, the at least one port 14d includes two ports 14d₁, 14d₂. One of the two ports is a blood port 14d₁ and is configured for filling and emptying the container 14b of the blood products. The other of the two ports, namely port 14d₂ may be a luer fitting port adapted to permit fluid to be introduced into the sampling bag 14 and/or to withdraw a portion of the blood product contained within the sampling bag 14. In either case, the port 14d enables a sterile connector for introducing or removing fluid from the sampling bag 14. In the depicted embodiment, the ports 14d₁, 14d₂ are located on one end of the sample container 14a. Each of the two ports 14d₁, 14d₂ may be located at a respective one of the short sides 14b of the sample container 14a. In the embodiment shown, the two ports 14d₁, 14d₂ are fluidly connectable to the internal volume V of the sample container 14a via an inlet 14e thereof.

As shown, the blood port 14d₁ includes a tube 18 of medical fluid flexible tubing. The tube 18 has a length extending from a first extremity 18a to a second extremity 18b. The first extremity 18a is secured to the sample container 14b and fluidly connected thereto and the second extremity 18b is fluidly connected to the main container 12. When the sample container 14a is not connected to the main container 12, the second extremity 18b of the tube 18 is closed or sealed.

Alternatively, the blood port 14d₁ may include a penetrable membrane that may be punctured by either one of a needle or a needleless medical grade fluid transfer fitting. Such fitting may include luer fittings and self-closing luer valves.

The main and sample containers 12, 14a are made of a plastic film, which may be plasticized polyvinyl chloride with a plasticizer. The main and sample containers may or may not be made of the same plastic film. The plasticizer may be a phthalate generally di(2-ethylhexyl) phthalate (DEHP), or alternatively an ester generally 1,2-cyclohexane-dicarboxylic acid diisononyl ester (DINCH), or alternatively a citrate generally n-butyryl-tri-n-hexyl citrate (BTHC). In a particular embodiment, the sample container 14a contains red blood cells and the plasticizer is DEHP. In a particular embodiment, the sample container 14a contains blood platelets and the plasticizer is BTHC. Other suitable plasticizers may however be used without departing from the scope of the present disclosure. Other materials are contemplated, for instance, other plastics which may or may not be PVC based might be used. Other suitable plastics and plasticizers may be used.

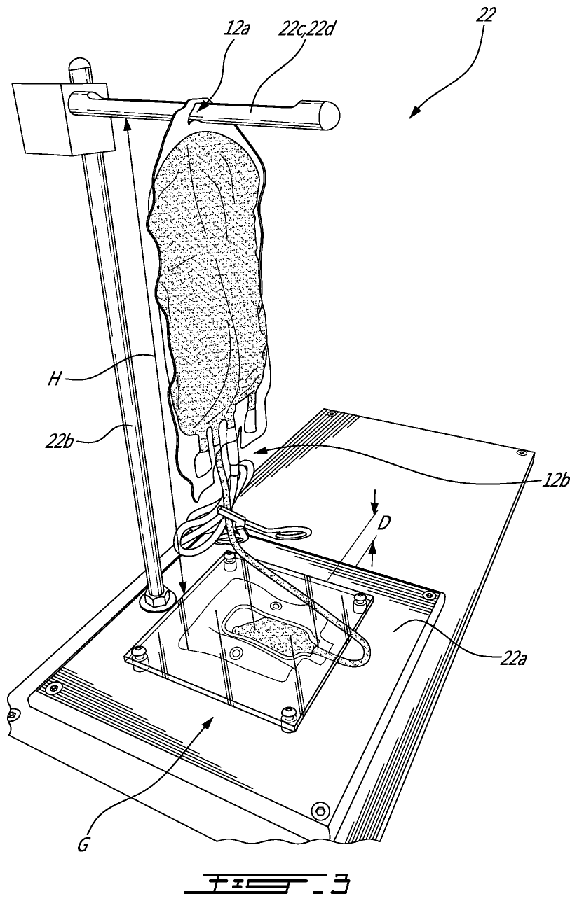

Referring to FIGS. 1 and 2, the sampling bag 14 includes a fixing mechanism 14f for securing in a sterile manner the sampling bag 14 to various filling, storage, and emptying apparatuses. The fixing mechanism 14f may also be used for securing the sampling bag to an agitating device 20 when the sampling bag 14 contains blood products, more specifically, and for example, platelets, that require intermittent or continuous agitation during their storage life. As shown, the fixing mechanism 14f includes flanges 14g each extending from a respective one of the long sides 14c of the container 14 and away therefrom. An aperture 14h, or a slit, is defined through each of the flanges 14g and is configured to be engaged by fixating pins 20a or holders of the agitating device 20. Other configurations are contemplated. A number and geometry of such aperture may be different and specific for specific product types. In a particular embodiment, two apertures, one directly opposed to the other, are used for platelets; and two apertures longitudinally offset from one another are used for red cells to facilitate identification and reduce risk of using incorrect containers.

As shown, the agitating device 20 includes a rack 20b having a plurality of apertures 20c defined therethrough. The pins 20a may be removably inserted in the apertures 20c at locations where they will register with the flange apertures 14h. The pins 14a may be screws, blocks, or hooks. The agitating device 20 may cause the rack to move in a linear, rocking, rotating, or oscillating manner. Preferably, when more than one sampling bag 14 is secured to the rack 20, the pins 20a are disposed within the rack apertures 20c such that the sampling bags 14 are spaced apart from each other; the sampling bags 14 preferably do not overlap each other. The fixating mechanism 14f allows for the agitating device 20 to transfer motion from the rack 20b, to the pins 20a, and to the sampling bags 14.

Referring now to FIGS. 1 and 3-4, a measuring, or filling, device for measuring a volume of blood products to be transferred in the sample container 14a, is generally shown at 22. As shown, the device 22 is gravity driven and includes a base 22a and a support arm 22b. The support arm 22b has a first end secured to the base and a second end vertically spaced apart from the first end and having a holder 22c for holding the main container 12 in an inverted position thereto at a given height H above the base 22a. In the embodiment shown, the holder 22c is a rod 22d configured to penetrate through an aperture 12a defined proximate an extremity of the main container 12 opposite a port 12b of the main container 12 for holding the main container 12 upside down so that gravity induces a flow of the blood product contained therein from the main container 12 toward the port 12b. The height D of the gap G may be controlled as a combination of head height H and constraining fixture plate offset D define the volume of product that will fill the test container 14a.

The measuring device 22 further includes a fixed volume constraining fixture 22e into which the sampling bag 14 is disposed for manual or automated filling. The fixed volume constraining fixture 22e is configured for limiting a volume of blood products transferred from the main container 12. The fixed volume constraining fixture 22e may be adjusted for varying a volume of blood products to be transferred from the main container 12 to the sample container 14a. In a particular embodiment, the fixture 22e is adjustable so that the volume of blood products may vary from 5 ml to 25 ml. In this particular embodiment, a sample container with a volume of blood product of less than 5 ml would not provide sufficient product for manipulation and testing and more than 25 ml might be problematic due to excessive volume (dose) depletion of the product in the main container, rendering it insufficient for transfusion purposes.

In the embodiment shown, the fixed volume constraining fixture 22e includes two plates 22f disposed parallel to one another and spaced apart from each other so as to define a gap G therebetween. In the embodiment shown, one of the two plates 22f is the base 22a and the other of the two plates 22f is disposed above the base 22a. The sample container 14a is disposed within the gap G and the distance D between the two plates 22f limits the volume of the blood products that may be transferred from the main container 12 to the sample container 14a. The distance D between the two plates 22f may be varied to increase or decrease a height of the gap G so that more or less blood products may be transferred to the sample container 14a from the main container 12. In the embodiment shown, the gap G defines the total volume of air and blood products to be contained in the sample container 14a. In the embodiment shown, the air volume is controlled by selecting a position for sterile docking on the tube 18 and the total volume of air and blood product contained in the sample container 14a is controlled by the height D of the gap G. The volume of blood product to be contained in the sample container 14a is defined by subtracting the volume of air to be inserted in the sample container 14a, which is determined by the docking position along the tube 18, from the total volume of air and blood product to be contained in the sample container 14a, which is determined by the height D of the gap G.

In the embodiment shown, spacers 22g are used for manually setting the distance D between the two plates 22f. Screws may alternatively be used. Alternatively, plungers or screws driven by compressed gas, electric coils, or electric motors may be used to move one of the plates 22f relative to the other to vary the distance D between the plates.

Alternatively, the fixed volume constraining fixture may also include a split cavity mold, wherein the size and/or shape of the cavity formed therein is predetermined and selected such that the sample container placed within the cavity can be filled with a predetermined (i.e. specified) volume of air and product. The measuring device may also be used for measuring a volume of air transferred in the sample container 14a. Accordingly, in certain embodiments, the measuring device is operable to control the introduction of just product into the sample container, and in other embodiments the measuring device is operable to control the introduction of both air and product into the container.

Referring to FIG. 7, a blocking device 22h, or a clamping mechanism, may be used to selectively allow or block a flow of the blood products from the main container 12 to the sample container 14a through the medical grade fluid flexible tube 18. In the embodiment shown, the clamping mechanism 22h is a hand-operated clamp 22i known in the field of transfusion and blood banking. Alternatively, the clamping mechanism 22h may be automated and use pinching blocks that may be driven by plungers or screws driven by compressed gas, electric coils, or electric motors.

For some blood products, such as blood platelets, it is preferable to maintain said products in movement, or in constant mixing, during their shelf life for proper preservation. It has been discovered that providing at least one air bubble within the sample container 14a may help in the mixing. The air bubble might affect/facilitate mixing for two different purposes: (1) during storage where needed (e.g. for platelet products) and (2) prior to removal from the sample container for testing.

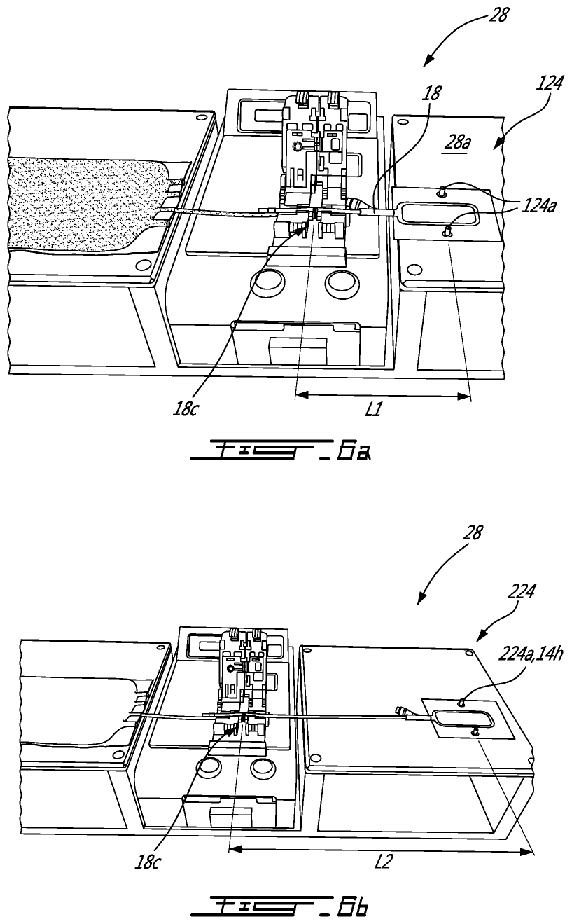

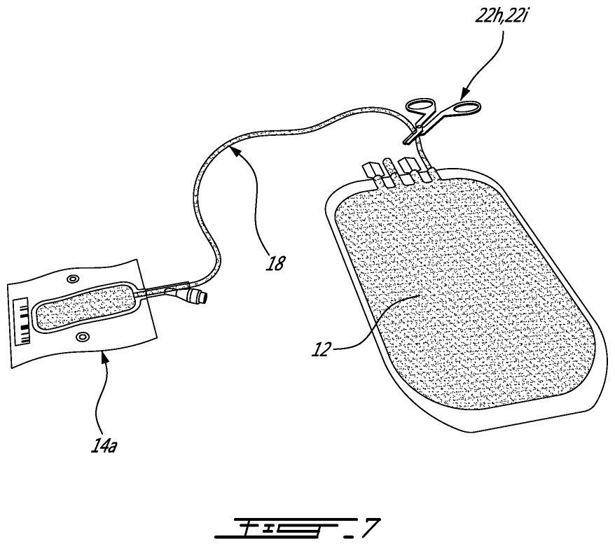

Referring now to FIG. 5, an apparatus for measuring a volume of air to be introduced into the sample container 14a during filling is generally shown at 24. As shown, the apparatus 24 is a measuring jig 26 used for creating an eye visible mark located on the medical flexible tube 18. The mark may be a line on the outer diameter of the tube 18. A distance between the mark and the inlet 14e of the sample container 14a is selected to indicate a location where the tube 18 is to be sterile docked to the main container 12 via an integrated sterile docking device 28 (FIGS. 6a-6b) that may be used with red blood cells (FIG. 6a) and with platelets (FIG. 6b).

A volume of air contained between the sample container inlet 14e and where the tube 18 is sterile docked is to be transferred in the sample container internal volume V during transfer of the blood products from the main container 12 to the sample container 14a. Hence, the greater the distance between the inlet and the where the tube 18 is docked the greater the volume of air in the sample container internal volume V. In the embodiment shown, the volume of air ranges from 0 ml to 5 ml and is function of which product of the blood products is to be transferred in the sample container 14a. For example, the volume of blood product in the sample container 14a may be from 5 to 25 ml. In one particular embodiment, therefore, the volume of the air bubble within the container corresponds to at least about 5% of the volume of the extracted blood product aliquot in the container. In a particular embodiment, the sample container contains 12 ml of platelets with about a 2 ml air bubble yielding a ratio of about 15%. As shown, the measuring jig defines a groove 26a for removably receiving the medical tube 18. The jig 26 is used for creating the eye visible marking described above.

Referring now to FIG. 6a, an apparatus to measure a volume of air to be introduced into the sample container 14a containing red blood cells during filling is generally shown at 124. The apparatus 124 includes pins 124a secured on a bench 28a of the docking device 28. A distance L1, taken along a longitudinal axis of the tube 18, extending from the pins 124a to a docking location 18c on the tube 18 is controlled to control the volume of air introduced in the container 14a. The locating pins 124a, which may alternatively be blocks fitting, are configured to register with either apertures 14h of the flanges 14g of the sample container 14a to hold the sample container 14a in a predetermined location.

Referring now to FIG. 6b, an apparatus to measure a volume of air to be introduced into the sample container 14a containing blood platelets during filling is generally shown at 224. The apparatus 224 includes pins 224a secured on the bench 28a of the docking device 28. A distance L2, taken along the longitudinal axis of the tube 18, extending from the pins 224a to the docking location 18c on the tube 18 is controlled to control the volume of air introduced in the container 14a. As shown, the distance L2 is greater than the distance L1 such that a volume of air introduced in a sample container 14a containing platelets is greater than a volume of air introduced in a sample container 14a containing red blood cells.

In a particular embodiment, the blood products are blood platelets, the sample container 14a has a capacity of 17 ml, the volume of air ranges from 1.5 ml to 2.5 ml, preferably 2 ml, and the volume of blood platelets ranges from 10 ml to 14 ml, preferably 12 ml. In a particular embodiment, the blood products are red blood cells; the sample container 14a having a capacity of 17 ml and containing from 8 to 10 ml, preferably 9 ml, of the blood products and from 0.5 to 1.5 ml of air, preferably 1 ml of air.

Referring to all Figures, all of the elements used for transferring the blood products from the main container 12 to the sample container 14a having been described, a method of transferring the blood products is now set forth.

The sample container 14a is fluidly connected in a sterile manner to the main container 12. A portion of the blood products is extracted from the main container 12 and transferred to the sample container 14a. A volume of air is provided in the sample container 14a with the extracted portion of the blood products. At least one air bubble 30 (FIG. 2) from the provided air is used for mixing, in the sample container 14a, the extracted portion of the blood products. A ratio of the volume of the air over the volume of the blood products in the sample container being about 0.02. The ratio of the volume of the air over the volume of the blood products may be 0 for some blood products. Obtaining a ratio of 0 might require use an automated filling device that ejects all residual air out of the sample container 14a. In the embodiment shown, transferring the extracted portion includes extracting from 0.25% to 20%, more specifically from 0.25% to 1.5%, of the volume of the blood products contained in the main container 12. In a particular embodiment, transferring the extracted portion includes extracting from 1% to 6% of the volume of the blood products contained in the main container 12. In the embodiment shown, transferring the extracted portion includes extracting from 5 ml to 25 ml of the volume of the blood products contained in the main container 12. In a particular embodiment, and depending on platelet product type, volumes can range from 200 ml to nearly 400 ml; similarly red cell concentrates would typically be in the 300 ml range.

As shown, fluidly connecting the sample container 14a to the main container 12 includes connecting the tube 18 to the main container 12 and releasing a blocking device 22h (FIG. 7) located on the tube 18 and between the sample container 14a and the main container 12; the blocking device 22h preventing the transfer of the blood products. The blocking device 22h may be, for instance, the clamp (FIG. 7) or an integral breakaway cannula located within the tube. The clamp may be an automated medical tubing clamp.

Alternatively, fluidly connecting the sample container 14a to the main container 12 includes providing the sample container 14a with the tube 18 having a length that may be sufficiently long to be sterile docked to a length of a medical grade fluid flexible tube secured to the main container 12. Alternatively, fluidly connecting the sample container 14a to the main container 12 includes providing the sample container with a needle and aseptically puncturing a penetrable membrane of the main container 12. Alternatively, fluidly connecting the sample container 14a to the main container 12 includes providing the sample container 14a with a needleless medical grade fluid transfer fitting including luer fittings and self-closing luer valves and attaching the needleless medical grade fluid transfer fitting to a mating fitting of the product.

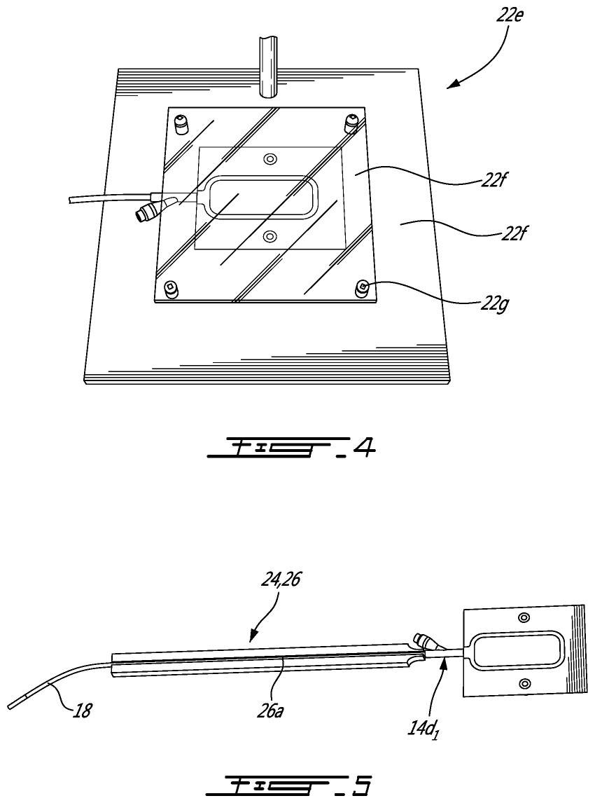

In the embodiment shown, extracting the portion of the blood products from the main container 12 includes inducing a flow of the blood products and controlling the volume of the blood products transferred from the main container 12 to the sample container 14a. As shown, inducing the flow of the blood products includes increasing a height of the main container 12 relative to the sample container 14a such that a gravity force is exerted on the blood products. In the embodiment shown, controlling the volume includes limiting a deformation of the sample container 14a upon filling beyond a predetermined level. In the embodiment shown, limiting the deformation includes disposing the sample container 14a between two plates 22f (FIG. 4); a distance D between the two plates 22f selected such that the two plates 22f exert a force on the sample container to limit further deformation of the sample container.

In the embodiment shown, providing the volume of air in the sample container 14a includes providing the sample container 14a with the volume of air before transferring the extracted portion of the blood products in the sample container 14a. In a particular embodiment, the sample container 14a is provided with the volume of air at the time of manufacture of the sample container 14a.

Alternatively, providing the volume of air includes transferring the volume of air from the medical grade flexible tube 18 to the sample container 14a; the tube 18 containing a pre-calculated volume of air. The volume of air is pushed from within the tube 18 in the sample container 14a by the blood products flowing from the main container 12 to the sample container 14a. Alternatively, providing the volume of air includes providing a means to measure the sterile docking location on a length of medical grade closed tubing attached to the sample container 14a which will contain a pre-calculated volume of air. The volume of air is pushed from within the tube 18 and into the sample container 14a by the blood products flowing from the main container 12 to the sample container 14a.

In the embodiment shown, providing the volume of air includes providing a volume of air varying from 0% to 25% of the volume of the main container 12. In the embodiment shown, providing the volume of air includes providing a volume of air varying from 0 ml to 5 ml of the volume of the main container 12. In a particular embodiment, the blood products are blood platelets, providing the volume of air includes providing the volume of air corresponding to at least 10% of the extracted portion of the blood products.

In the embodiment shown, using the at least one air bubble 30 for mixing further includes securing the sample container 14a to the agitating device 20; and moving the at least one air bubble 30 via the agitating device 20. As shown, securing the sample container 14a to the agitating device 20 includes securing the flanges 14g of the sampling bag 14 to the rack 20b of the agitating device 20 via registering apertures 14h and fixing pins 20a.

In the embodiment shown, the main container 12 is manually or automatically mixed for homogeneity before fluidly connected the sample container 14a to the main container 12.

In the depicted embodiment, the sample container 14a is identified by providing an eye or machine readable identifier 32 (FIG. 2); the identifier 32 indicating of a unique donation or other product number. The identifier 32 may be hand written or machine printed on the flange 14g of the sampling bag 14. The identifier 32 is used to provide traceability of the blood product contained within the sample container 14a. The identifier 32 may be a bar code printed onto an adhesive label applied to the flange 14g or printed directly thereon. Alternatively, a RFID tag may be embedded within the flange 14g, on an adhesive label or applied elsewhere on the sample container.

The main container 12 and the sample container 14a may be separated after transferring the blood products from the main container 12 to the sample container 14a. As shown, this step may be performed by disconnecting a self-sealing aseptic connection located between the sample container and the main container. The self-sealing aseptic connecting may be a self-sealing puncturable membrane or luer fitting valves, the disconnection may include removing a needle or a luer from the puncturable membrane or the luer fitting valves. Alternatively, the disconnection includes sealing and cutting the tube 18 via either heat, high frequency tubing sealers, a combination thereof, or any suitable method known in the art.

The method further includes transferring the extracted portion of the blood products out of the sample container 14a for carrying out specific quality attribute tests at a specific time. The specific time may be up to and beyond time of product expiry. In the embodiment shown, the fluid is withdrawn using a syringe attached to the needleless access port. Transferring the extracted portion out of the sample container 14a may include puncturing the tube 18 by a needle and drawing the extracted portion of the blood products out of the sample container 14a. Alternatively, the sample container 14a may be cut to allow the blood products to be drained out of the sample container 14a. Alternatively, a penetrable membrane which may be punctured by a needle to draw product out of the sample container 14a, or alternatively a needle through which product could be drawn out of the sample container 14a into an evacuated container, or alternatively a needleless medical grade fluid transfer fitting including luer fitting and self-closing luer valve to which a syringe or evacuated container transfer device may be attached and into which product could be drawn from the sample container.

For sampling an aliquot of the blood products from a main container containing the blood products to be sampled includes: providing the sample container that is rectangular-shaped and has a length-over-width (L/W) ratio of at least that of the main container, and fluidly connecting a sample container to the main container; transferring the aliquot of the blood products from the main container to the sample container; and introducing a volume of air corresponding to at least about 5% of a volume of the aliquot of the blood products in the sample container with the aliquot of the blood products to form an air bubble in the sample container.

In the embodiment shown, a tube is used to fluidly connect the sample container to the main container; introducing the volume of air includes injecting air contained within the tube into the sample container. Herein, injecting the air includes transferring the air with the blood products from the main container to the sample container via the tube. In the embodiment shown, introducing the volume of air includes anchoring the sample container at a given distance from the docking location at which the sample container is fluidly connected to the main container via the tube. The tube is fluidly connected to the sample container. In the embodiment shown, anchoring the sample container at the given distance includes inserting at least one locking pin secured to a bench supporting the sample container inside at least one aperture defined through a flange of the sample container. As illustrated, inserting the at least one locking pin through the at least one aperture includes inserting two locking pins through two apertures defined through the flange.

In the depicted embodiment, transferring the aliquot of the blood products includes filling the sample container until the sample container becomes compressed between two spaced apart plates. The sample container may be disposed between the two spaced apart plates.

A correlation between a distance between the two spaced apart plates and a maximum volume of fluid containable within the sample container located between the two spaced apart plates is determined. This correlation may be determined, for instance, by experimental testing.

In the embodiment shown, transferring the aliquot of the blood products includes drawing the blood products out of the main container by gravity. The main container may be hanged above the sample container.

In some cases, the extracted portion of the blood products is mixed in the sample container using the air bubble. The blood products may be blood platelets, transferring the extracted portion includes transferring the extracted aliquot in the sample container having the length-over-width (L/W) ratio of about 3.

Introducing the volume of air in the sample container may include providing the sample container with the volume of air therein before transferring the extracted aliquot in the sample container.

The at least one air bubble may be moved within the sample container by agitating the sample container.

In a particular embodiment, transferring the aliquot of the blood products includes extracting at most 20% of a volume of the blood products contained within the main container. The blood products may be blood platelets, providing the volume of air includes providing the volume of air corresponding to at least 15% of the volume of the extracted aliquot of the blood products.

In a particular embodiment, the contents of sample container is mixed to ensure homogeneity. This might be aided by the presence of air bubble in the container 14a if removing partial volume. An air bubble may be used for mixing the content of the sample container, whether said content is blood platelets or red blood cells.

The content of the main container may be released for transfusion after sampling. The sample bag 14 may be stored in appropriate environment for later testing. A portion or all of the volume from the sample container 14 may be removed for testing at some later time up to and beyond expiry of the product in the main container. Results obtained from analysis of the test container may be correlatable to results that would be obtained from the main container if tested at the same time point.

In some cases, an air bubble is not required. The air bubble may be present to aid in mixing during agitation when required for a particular blood product like platelets and/or to facilitate good mixing of the blood component in the test container 14 to help ensuring homogeneity prior to removing a portion for testing.

In a particular embodiment, the disclosed sampling system 10 is non-destructive and enables units selected for process control to be sampled early in their shelf life and subsequently released to inventory for issue to hospitals for transfusion purposes. This sampling system might allow for quality attribute testing of the aliquot at any time point up to and including the date of expiry, and beyond expiry date, of the main container 12 from which the sample was removed, and where the quality attributes of the aliquot are known to be representative at the time of testing of the main container 12 from which it was removed. In a particular embodiment, testing is done on the day after expiry. This sampling system 10 might allow for increasing the process control sample size to any level up to and including 100% of a total volume of blood products produced without impacting product available for inventory and issue to hospitals. This sampling system might allow for targeted sampling of specific containers, as for example, for the purposes of donor qualification or donor factor studies.

Embodiments disclosed herein include:

A. A sampling bag for holding blood products comprising: a sample container defining therein an internal volume adapted to contain the blood products, the sample container made of a flexible plastic film and having a rectangular shape defined by short sides and long sides, the short sides and the long sides delimiting the internal volume of the container, the long sides having a length (L) and the short sides having a width (W), wherein a ratio (L/W) of the length over the width is at least 1.5; and at least one port secured to one of the long sides or to one of the short sides, the at least one port fluidly connectable to a source of the blood products for inserting the blood products into the internal volume of the container.

Embodiment A may include any of the following Elements, in whole or in part, and in any combination:

Element 1: the ratio (L/W) is at most 10. Element 2: the blood products are platelets, and the ratio (L/W) is about 3. Element 3: the plastic film is made of plasticized polyvinyl chloride. Element 4: the blood products are platelets, and a plasticizer of the plastic film being a citrate. Element 5: the citrate is n-butyryl-tri-n-hexyl citrate. Element 6: the sampling bag includes a fixing mechanism for securing the sampling bag to an agitating device. Element 7: the internal volume ranges from 5 ml to 25 ml. Element 8: the at least one port includes a tube of medical fluid flexible tubing, the tube having a length extending from a first extremity to a second extremity, the first extremity being secured to the container and fluidly connected thereto, the second extremity being configured for being fluidly connected to a main container of the blood products.

B. A sampling system for sampling blood products, comprising: a main container configured for containing a first volume of the blood products; a sampling bag having a sample container made of a plastic film and configured for containing a second volume of the blood products, the sample container having a rectangular shape and having a length-over-width (LAN) ratio of at least that of the main container, the second volume being less than the first volume; and a fluid connection between the main container and the sample container.

Embodiment B may include any of the following Elements, in whole or in part, and in any combinations:

Element 10: further comprising a filling device for measuring the second volume, the filling device including two places being spaced apart from one another by a gap, the sample container within the gap, a maximum volume of fluid contained in the sample container being defined by a height of the gap. Element 11: the filling device includes a support arm extending transversally relative to the plates, the support arm having a holder above the spaced apart plates for holding the main container above the sample container. Element 12: further comprising an apparatus for measuring a volume of air to be inserted in the sample container, the apparatus having: a bench supporting the sample container; a sterile docking device for fluidly connecting the sample container to the main container via a tube; and at least one pin protruding from the bench, the at least one pin received within at least one aperture defined through a flange of the sample container, wherein a distance along a length of the tube from the at least one locking pin to a docking location of the sterile docking device is selected such that a volume of air contained within the tube along the distance corresponds to the volume of air to be inserted in the sample container. Element 13: the blood products are blood platelets, the ratio of the length over the width being about 3. Element 14: the plastic film is made of plasticized polyvinyl chloride. Element 15: a ratio of the second volume over the first volume is at most 0.2.

C. A method of sampling an aliquot of blood products from a main container containing the blood products to be sampled, the method comprising: providing a sample container that is rectangular-shaped and has a length-over-width (L/W) ratio of at least that of the main container, and fluidly connecting the sample container to the main container; transferring the aliquot of the blood products from the main container to the sample container; and forming an air bubble in the sample container by introducing a volume of air into the sample container with the aliquot of the blood products, the volume of air forming the air bubble corresponding to at least about 5% of a volume of the aliquot of the blood products.

Embodiment C may include any of the following elements in any combinations:

Element 20: injecting the air includes transferring the air with the blood products from the main container to the sample container via the tube. Element 21: introducing the volume of air includes anchoring the sample container at a given distance from a docking location at which the sample container is fluidly connected to the main container via the tube. Element 22: further comprising fluidly connecting the tube to the sample container. Element 23: anchoring the sample container at the given distance includes inserting at least one locking pin secured to a bench supporting the sample container inside at least one aperture defined through a flange of the sample container. Element 24: inserting the at least one locking pin through the at least one aperture includes inserting two locking pins through two apertures defined through the flange. Element 25: transferring the aliquot of the blood products includes filling the sample container until the sample container becomes compressed between two spaced apart plates. Element 26: further comprising disposing the sample container between the two spaced apart plates. Element 27: further comprising determining a correlation between a distance between the two spaced apart plates and a maximum volume of fluid containable within the sample container located between the two spaced apart plates. Element 28: transferring the aliquot of the blood products includes drawing the blood products out of the main container by gravity. Element 29: further comprising hanging the main container above the sample container. Element 30: further comprising mixing the extracted portion of the blood products in the sample container using the air bubble. Element 31: the blood products are blood platelets, transferring the extracted portion includes transferring the extracted aliquot in the sample container having the length-over-width (L/W) ratio of about 3. Element 32: introducing the volume of air in the sample container includes providing the sample container with the volume of air therein before transferring the extracted aliquot in the sample container. Element 33: further including moving the at least one air bubble within the sample container by agitating the sample container. Element 34: transferring the aliquot of the blood products includes extracting at most 20% of a volume of the blood products contained within the main container. Element 35: the blood products are blood platelets, providing the volume of air includes providing the volume of air corresponding to at least 15% of the volume of the extracted aliquot of the blood products. Element 36: further comprising using a tube to fluidly connect the sample container to the main container, wherein introducing the volume of air includes injecting air contained within the tube into the sample container.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. A method of sampling an aliquot of blood products from a main container containing the blood products to be sampled, the method comprising:
providing a sample container that is rectangular-shaped and has a length-over-width (L/W) ratio of at least that of the main container, and fluidly connecting the sample container to the main container;
transferring the aliquot of the blood products from the main container to the sample container; and
forming an air bubble in the sample container by introducing a volume of air into the sample container with the aliquot of the blood products, the volume of air forming the air bubble corresponding to at least about 5% of a volume of the aliquot of the blood products.

2. The method of claim 1, further comprising using a tube to fluidly connect the sample container to the main container, wherein introducing the volume of air includes injecting air contained within the tube into the sample container.

3. The method of claim 2, wherein injecting the air includes transferring the air with the blood products from the main container to the sample container via the tube.

4. The method of claim 3, wherein introducing the volume of air includes anchoring the sample container at a given distance from a docking location at which the sample container is fluidly connected to the main container via the tube.

5. The method of claim 4, further comprising fluidly connecting the tube to the sample container.

6. The method of claim 4, wherein anchoring the sample container at the given distance includes inserting at least one locking pin secured to a bench supporting the sample container inside at least one aperture defined through a flange of the sample container.

7. The method of claim 6, wherein inserting the at least one locking pin through the at least one aperture includes inserting two locking pins through two apertures defined through the flange.

8. The method of claim 1, wherein transferring the aliquot of the blood products includes filling the sample container until the sample container becomes compressed between two spaced apart plates.

9. The method of claim 8, further comprising disposing the sample container between the two spaced apart plates.

10. The method of claim 8, further comprising determining a correlation between a distance between the two spaced apart plates and a maximum volume of fluid containable within the sample container located between the two spaced apart plates.

11. The method of claim 1, wherein transferring the aliquot of the blood products includes drawing the blood products out of the main container by gravity.

12. The method of claim 11, further comprising hanging the main container above the sample container.

13. The method of claim 1, further comprising mixing the extracted portion of the blood products in the sample container using the air bubble.

14. The method of claim 1, wherein the blood products are blood platelets, transferring the extracted portion includes transferring the extracted aliquot in the sample container having the length-over-width (L/W) ratio of about 3.

15. The method of claim 1, wherein introducing the volume of air in the sample container includes providing the sample container with the volume of air therein before transferring the extracted aliquot in the sample container.

16. The method of claim 1, further including moving the at least one air bubble within the sample container by agitating the sample container.

17. The method of claim 1, wherein transferring the aliquot of the blood products includes extracting at most 20% of a volume of the blood products contained within the main container.

18. The method of claim 1, wherein the blood products are blood platelets, providing the volume of air includes providing the volume of air corresponding to at least 15% of the volume of the extracted aliquot of the blood products.

19. A sampling bag for holding blood products comprising:
a sample container defining therein an internal volume adapted to contain the blood products, the sample container made of a flexible plastic film and having a rectangular shape defined by short sides and long sides, the short sides and the long sides delimiting the internal volume of the container, the long sides having a length (L) and the short sides having a width (W), wherein a ratio (L/W) of the length over the width is at least 1.5; and
at least one port secured to one of the long sides or to one of the short sides, the at least one port fluidly connectable to a source of the blood products for inserting the blood products into the internal volume of the container.

20.-27. (canceled)

28. A sampling system for sampling blood products, comprising:
a main container configured for containing a first volume of the blood products; a sampling bag having a sample container made of a plastic film and configured for containing a second volume of the blood products, the sample container having a rectangular shape and having a length-over-width (L/W) ratio of at least that of the main container, the second volume being less than the first volume; and
a fluid connection between the main container and the sample container.

29.-34. (canceled)
